(19) United States
(12) Patent Application Publication
(10) Pub. No.: US 2026/0325213 A1
(43) Pub. Date: Oct. 22, 2026

(54) SYSTEM FOR GENERATION AND ADAPTATION OF RESECTION VOLUME

(71) Applicant: GE Precision Healthcare LLC, Waukesha, WI (US)

(72) Inventors: Amalie Ekstrand, Copenhagen (DK); Jacob Bjerring Olesen, Copenhagen (DK); Bo Martins, Roedovre (DK); Mads Andersen, Copenhagen (DK)

(73) Assignee: GE Precision Healthcare LLC, Waukesha, WI (US)

(21) Appl. No.: 19/182,494

(22) Filed: Apr. 17, 2025

(51) Int. Cl. A61B 34/10; A61B 8/00; A61B 8/08

(52) U.S. Cl. CPC A61B 34/10; A61B 8/085; A61B 8/466; A61B 8/483; A61B 2034/105; A61B 2034/107

(57) ABSTRACT

An ultrasound imaging system includes a transducer configured to transmit a transmit signal and receive an ultrasound signal. The system includes a processing circuit having a processor coupled to a memory device storing instructions thereon that, when executed, cause the processing circuit to perform operations including generating a three-dimensional (3D) scene based on the ultrasound signal including a first anatomical feature, determining a position of an abnormality of the first anatomical feature based on the ultrasound signal, generating and displaying a resection volume on the first anatomical feature within the 3D scene based on the position of the abnormality, determining the resection volume interferes with a second anatomical feature, and adapting the resection volume based on the determination that the planned resection volume interferes with the second anatomical feature. The operations include displaying an output image. The output image includes the 3D scene including the adapted planned resection volume.

### FIELD

Embodiments of the subject matter disclosed herein relate to ultrasound imaging, and more particularly, to the use of ultrasound imaging for generation and adaptation of a resection volume.

### BACKGROUND

Surgical resection is used for curative care of abnormalities (e.g., primary and metastatic hepatic tumors). A surgical resection can be anatomical, where the entire segment or subsegment in which the abnormality is located is removed, or non-anatomical, where only the tumor and a safety margin around it is removed. Laparoscopic ultrasound is currently used to guide the resection line planning for non-anatomical resections. A surgeon scans the area around the tumor and draws resection lines. The placement of the resection lines rely on the surgeon being able to reconstruct the information from the two-dimensional ultrasound images into a three-dimensional scene over vessels surrounding the abnormality. This is especially difficult during a laparoscopic surgery, where the visual information of the probe placement is also limited. As a result, there may be difficulties with locating the abnormality and identifying vessels to avoid in the vicinity of the abnormality.

### SUMMARY

An embodiment relates to an ultrasound imaging system. The ultrasound imaging system includes a transducer configured to transmit a transmit signal and receive an ultrasound signal. The ultrasound imaging system also includes a processing circuit. The processing circuit includes a processor coupled to a memory device storing instructions thereon that, when executed, cause the processing circuit to perform operations including generating a three-dimensional (3D) scene based on the ultrasound signal including a first anatomical feature, positioning an abnormality of the first anatomical feature in the 3D scene based on the ultrasound signal, generating and displaying a resection volume on the first anatomical feature within the 3D scene based on the position of the abnormality, determining the resection volume interferes with a second anatomical feature, and adapting the resection volume based on the determination that the planned resection volume interferes with the second anatomical feature. The operations include displaying an output image on a display screen of the ultrasound imaging system. The output image includes the 3D scene including the adapted planned resection volume.

Another embodiment relates to an ultrasound imaging system. The ultrasound imaging system includes a transducer configured to transmit a transmit signal and receive an ultrasound signal. The ultrasound imaging system includes an image processing circuit configured to generate a 3D scene based on the ultrasound signal including a first anatomical feature and position an abnormality of the first anatomical feature. The ultrasound imaging system includes a control circuit configured to generate and display a resection volume on the first anatomical feature within the 3D scene based on the position of the abnormality and upon determining the planned resection volume interferes with a second anatomical feature, and adapting the resection volume. The ultrasound imaging system is configured to display an output image on a display screen of the ultrasound imaging system. The output image includes the 3D scene including the adapted planned resection volume.

Another embodiment relates to a method. The method includes generating, by a processing circuit, a 3D scene based on an ultrasound signal. The 3D scene includes a first anatomical feature. The method includes positioning, by the processing circuit, a an abnormality of the first anatomical feature in the 3D scene based on the ultrasound signal. The method includes generating and displaying, by the processing circuit, a resection volume on the first anatomical feature within the 3D scene based on the position of the abnormality. The method includes determining, by the processing circuit, the resection volume interferes with a second anatomical feature. The method includes adapting, by the processing circuit, the resection volume based on the determination that the planned resection volume interferes with the second anatomical feature. The method includes displaying an output image on a display screen of an ultrasound imaging system. The output image includes the 3D scene including the adapted planned resection volume.

This summary is illustrative only and is not intended to be in any way limiting. Other aspects, inventive features, and advantages of the devices or processes described herein will become apparent in the detailed description set forth herein, taken in conjunction with the accompanying figures, wherein like reference numerals refer to like elements.

### BRIEF DESCRIPTION OF THE DRAWINGS

FIG. 1 is a block diagram of an ultrasound imaging system, according to an example embodiment.

FIG. 2 is an illustration of scan planes, according to an example embodiment.

FIG. 3 is an illustration of a resection volume, according to an example embodiment.

FIG. 4 is a flow chart illustrating a method for resection planning, according to an example embodiment.

FIG. 5 is a flow chart illustrating a method for resection planning, according to an example embodiment.

FIG. 6 is a flow chart illustrating a method for ultrasound imaging using the ultrasound imaging system of FIG. 1, according to an example embodiment.

FIG. 7A an illustration of an anatomical feature including a planned resection volume, according to an example embodiment.

FIG. 7B an illustration of the anatomical feature of FIG. 7A including an adapted planned resection volume, according to an example embodiment.

FIG. 8A an illustration of an anatomical feature including a planned ablation, according to an example embodiment.

FIG. 8B an illustration of the anatomical feature of FIG. 8A including an adapted planned ablation, according to an example embodiment.

### DETAILED DESCRIPTION

Referring generally to the figures, systems and methods for the use of ultrasound imaging for generation and adaptation of a resection volume are disclosed. The systems disclosed herein are used to generate a three-dimensional (3D) scene based on an ultrasound signal. The systems disclosed herein use the 3D scene to generate and display a planned resection volume on a first anatomical feature within the 3D scene. The systems disclosed herein are used to determine the resection volume interferes with a second anatomical feature and adapt the resection volume based on the determination. The systems and methods use the 3D scene such that a display screen may display an output image of the 3D scene including the adapted planned resection volume.

During a surgical resection, ultrasound imaging (e.g., laparoscopic ultrasound) is used to guide rection line planning. Ultrasound imaging is used to locate an abnormality to be resected (e.g., a tumor), determine an extension of the abnormality, and to identify nearby anatomical features in proximity to the abnormality. For example, for a liver resection in which an abnormality is within the liver, a surgeon make take an ultrasound scan of the liver and an area around the abnormality and draw resection lines on a surface of the liver to mark the resection and plan a resection volume. Placement of the resection lines rely on the skills and expertise of the surgeon (or operator, sonographer, technician, clinician, etc.), thereby resulting in inconsistencies of resection lines, tissue resections, etc., due to surgeon efficiencies. The surgeon is required to reconstruct information from two-dimensional (2D) images into a 3D scene to capture information on surrounding anatomical features. This may be especially difficult during a laparoscopic surgery where visual information of the probe placement is also limited. Additional difficulties may arise as the liver may move around and adjust within the body, such that the position of the tumor and the resection volume may change between the determination of the resection volume and the resection procedure.

Additionally, the planning of the resection volume is generally estimated during planning of the surgery and is based on preoperative data. However, the preoperative data may not include the most updated information of the anatomical feature and the abnormality. For example, with the liver resection, the liver may have deformed from the time between the preoperative data being gathered and the liver resection. As a result, the planned resection volume may no longer be optimal based on the current anatomy of the liver.

The systems and methods described herein provide a technical solution to existing resection methods through the usage of ultrasound imaging systems. The use of ultrasound imaging allows for a 3D scene to be obtained to visualize a first anatomical feature and an abnormality and for resection volume to be analyzed to determine whether it interferes with a second anatomical feature. This differs from current methods in which reconstructing information received from ultrasound imaging into a 3D scene depended on a skill of the operator. Additionally, the systems and methods described herein allow for the resection volume to be determined based on live imaging and while taking critical structures (e.g., the second anatomical feature) into account). The position of the second anatomical feature may impact an angle of entry during the resection, resulting in a different resection volume than what was expected from the preoperative data. As used herein, the angle of entry refers to the angle at which a medical instrument (e.g., needle) is inserted into the patient’s body. By live updating the resection strategy during the procedure with the systems and methods described herein, a more accurate estimate of the resection volume is generated. The 3D scene obtained through ultrasound imaging can be used to determine and adapt a resection volume such that the second anatomical feature does not interfere with the resection volume. The systems and methods described herein can be used to obtain a visualization of a resection volume and ensure that viable tissue is not being resected.

Thus, the systems and methods described herein reduce the dependency on the expertise and skills of the operator by automatically generating a 3D scene based on the ultrasound signal. Additionally, the system may automatically adapt the resection volume based on a determination that the planned resection volume interferes with a second anatomical feature, thereby reducing the need for the operator to maneuver the probe manually to find a desirable resection volume. Furthermore, the systems and methods described herein assist an operator in generating a resection volume and completing an ultrasound exam in a shorter amount of time (e.g., due to the operator not having to spend time reconstructing images into a 3D scene).

The implementations described herein address a technical problem by providing enhanced data integration and analysis capabilities, which deliver a particular technical solution that streamlines and refines generating a 3D scene for information during a laparoscopic ultrasound. The systems described herein are implemented to improve how data is synthesized and utilized from various sources that provide information relating to determination and assessment of an abnormality and a resection volume. Accordingly, the approaches disclosed herein provide a specific technical improvement to various technical problems, including those set forth herein. The systems described herein may also reduce processing power by performing various processing operations simultaneously to generate images during an ultrasound scan, rather than performing a plurality of processing operations individually and consuming unnecessary processing power.

Before turning to the figures, which illustrate certain exemplary embodiments in detail, it should be understood that the present disclosure is not limited to the details or methodology set forth in the description or illustrated in the figures. It should also be understood that the terminology used herein is for the purpose of description only and should not be regarded as limiting.

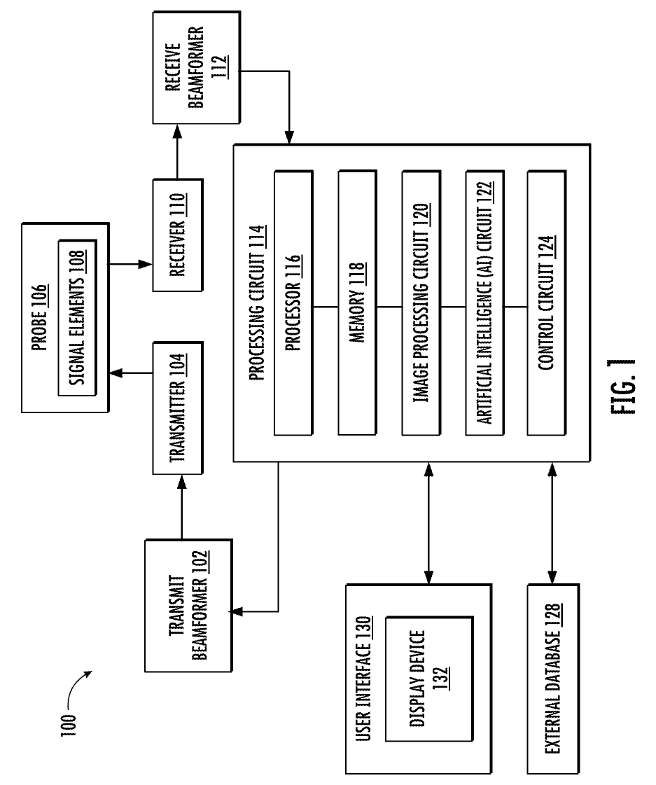

Referring to FIG. 1, a schematic diagram of an ultrasound imaging system 100 is shown. The ultrasound imaging system 100 may be used in a medical environment (e.g., hospitals, clinics, etc.), for example, by a sonographer, technician, or other clinician certified to collect ultrasound data from a patient.

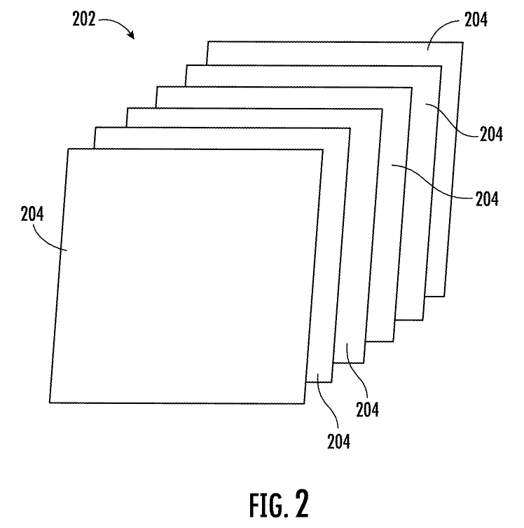

An example of a procedure performed using the ultrasound imaging system 100 may be a laparoscopic ultrasound. The laparoscopic ultrasound may be performed to detect various pathologies and conditions such as liver tumors, cysts, abscesses, vascular abnormalities, gallbladder issues, gallstones, and so on. Such pathologies are detected by collecting and processing ultrasound data (e.g., using the ultrasound imaging system 100, as described herein). During the laparoscopic ultrasound, a sonographer collects the ultrasound data by navigating a probe (e.g., probe 106, as described below) over a patient’s chest until a sufficient volume of ultrasound images are collected. As described herein with reference to FIG. 2, a collection of 2D images can be collected via two-dimension imaging to form a 3D scene. With three-dimensional imaging, using the ultrasound imaging system 100, a three-dimension volume of ultrasound images may be obtained. The sonographer may collect the ultrasound images both with the ultrasound probe in a sagittal orientation, a transverse orientation, and, with three-dimensional imaging, a coronal orientation. With reference to the Cartesian coordinate system described herein, the transverse orientation refers to the XZ plane or the YZ plane. The sagittal orientation refers to the other of the XZ plane or the YZ plane. The coronal orientation refers to the XY plane. The collected images are stored in a central storage device (e.g., memory 118) and analyzed by the sonographer. The sonographer generates a set of measurements from the images (e.g., 50-100 records), and the images and measurements are collectively reviewed by a medical expert, such as a pulmonologist. The pulmonologist provides any clinical findings/conclusions in a report submitted to the patient’s medical record.

As shown in FIG. 1, the ultrasound imaging system 100 includes a transmit beamformer 102, a transmitter 104, a probe 106, a receiver 110, and a receive beamformer 112. The ultrasound imaging system 100 may also include a matching layer and a damping block. The matching layer improves ultrasound image quality by having an acoustic impedance between a tissue to be imaged and a material of a transducer of the ultrasound imaging system 100. The damping block is configured to absorb ultrasound energy to improve ultrasound image quality.

The transmit beamformer 102 may be either a hardware beamformer or a software beamformer. In embodiments where the transmit beamformer 102 is a hardware beamformer, the transmit beamformer 102 may include one or more of a graphics processing unit (GPU), a microprocessor, a central processing unit (CPU), a digital signal processor (DSP), or any other type of processor capable of performing logical operations. The transmit beamformer 102 may be configured to perform conventional beamforming techniques as well as techniques such as retrospective transmit beamforming (RTB). Alternatively, in embodiments where the transmit beamformer 102 is a software beamformer, a processor (e.g., processor 116, as described below) may be configured to perform some or all of the functions associated with the transmit beamformer 102.

The probe 106 may be a linear array probe, a curvilinear array probe, a sector probe, a row-column array probe, or any other type of probe configured to obtain ultrasound data (e.g., B-mode data, color flow data, etc.). More specifically, the probe 106 may be any type of probe including a matrix transducer array configured to obtain three-dimensional (3D) or two-dimensional (2D) ultrasound data. In some embodiments, the probe 106 may include a position sensor configured to detect a position of the probe 106 relative to one or more reference locations. That is, the position sensor may continuously track movement (e.g., rotation, translation, orientation, etc.) of the probe 106 relative to the location of the probe 106 when the anatomy being imaged is identified. For example, the anatomy being imaged may be identified as a liver (e.g., represented by liver 700, as described below) at a first location of the probe 106. Then, the position sensor may track the movement of the probe 106 relative to the liver to identify successive locations of the probe 106. In some embodiments, the position sensor may transmit position data to be stored within the ultrasound imaging system 100 (e.g., in memory 118).

The probe 106 may include a transducer configured to transmit and receive an ultrasound signal. In some embodiments, as shown in FIG. 1, the probe 106 includes signal elements 108. The signal elements 108 may be arranged in a transducer array, and in some embodiments may be arranged in a 2D array. As described herein, the 2D array of the signal elements 108 may allow for 3D ultrasound imaging (e.g., such that the transducer is configured to transmit and receive a 3D ultrasound signal). The transmit beamformer 102 and the transmitter 104 drive the signal elements 108 to emit pulsed ultrasonic signals into a body of a subject (e.g., a patient). For example, during a laparoscopic ultrasound, a sonographer or other clinician may navigate the probe 106 over a patient’s chest so that the signal elements 108 in the probe 106 emit the pulsed ultrasonic signals into the patient’s abdominal cavity. The pulsed ultrasonic signals are then back-scattered from anatomical structures in the body, such as blood cells or muscular tissues, to produce echoes that return to the signal elements 108.

Additionally, in ultrasound imaging systems with electronically steered beams, the transmit beamformer 102 may be adjusted to shift an angle of the ultrasound beam, therefore shifting the ultrasound signal transmitted. For example, the transmit beamformer 102 may adjust timing and phase shifts of the ultrasound signals sent into the body of the subject to change the angle of the ultrasound signal. In various embodiments, the adjustment may occur without the operator having to move the transducer.

The receiver 110 receives the echoes from the probe 106 and converts the echoes into electrical signals. The electrical signals are then passed through the receive beamformer 112, which produces the ultrasound data from the electrical signals. As described above with reference to the transmit beamformer 102, the receive beamformer 112 may be either a hardware beamformer or a software beamformer. In embodiments where the receive beamformer 112 is a hardware beamformer, the receive beamformer 112 may include one or more of a GPU, a microprocessor, a CPU, a DSP, or any other type of processor capable of performing logical operations. The receive beamformer 112 may be configured to perform conventional beamforming techniques as well as techniques such as retrospective transmit beamforming (RTB). Alternatively, in embodiments where the receive beamformer 112 is a software beamformer, a processor (e.g., processor 116, as described below) may be configured to perform some or all of the functions associated with the receive beamformer 112.

Although the transmit beamformer 102, the transmitter 104, the receiver 110, and the receive beamformer 112 are shown in FIG. 1 as being components of the ultrasound imaging system 100 that are distinct from the probe 106, it should be appreciated that in some embodiments, the probe 106 may include electronic circuitry configured to perform the functions of each of the transmit beamformer 102, the transmitter 104, the receiver 110, and/or the receive beamformer 112. That is, all or part of the transmit beamformer 102, the transmitter 104, the receiver 110, and/or the receive beamformer 112 may be situated within the probe 106.

Referring still to FIG. 1, the ultrasound imaging system 100 is shown to include a processing circuit 114. As shown, the processing circuit 114 may include at least one processor 116, a memory 118, an image processing circuit 120, an artificial intelligence (AI) circuit 122, and a control circuit 124. In this way, the processing circuit 114 may be structured or configured to execute or implement the instructions, commands, and/or control processes described herein with respect to the processor 116, the memory 118, the image processing circuit 120, the AI circuit 122, and the control circuit 124. While shown as being separate from the probe 106 in FIG. 1, it will be appreciated that the processing circuit 114 can be part of the probe 106. For example, the processing circuit 114 can be disposed in a handheld housing of the probe 106 (e.g., in the case of the probe 106 being a wireless probe).

The processor 116 may include a CPU, a GPU, a microprocessor, a DSP, a general-purpose single- or multi-chip processor, a field-programmable gate array (FPGA), or any other type of processor capable of performing logical operations. A general-purpose processor may be a microprocessor, or, any conventional processor, or state machine. A processor also may be implemented as a combination of computing devices, such as a combination of a DSP and a microprocessor, a plurality of microprocessors, one or more microprocessors in conjunction with a DSP core, or any other such configuration. In some embodiments, the processor 116 may be shared by multiple circuits (e.g., the circuits of the processor 116 may include or otherwise share the same processor which, in some example embodiments, may execute instructions stored, or otherwise accessed, via different areas of the memory 118). Alternatively or additionally, the processor 116 may be structured to perform or otherwise execute certain operations independent of one or more co-processors. In some embodiments, two or more processors may be coupled via a bus to enable independent, parallel, pipelined, or multi-threaded instruction execution. All such variations are intended to fall within the scope of the present disclosure.

The processor 116 may be configured to control the transmit beamformer 102, the transmitter 104, the receiver 110, and the receive beamformer 112. The processor 116 may also be in electronic communication with the probe 106. For purposes of this disclosure, the term “electronic communication” may be defined to include both wired and wireless communications.

In some embodiments, the processor 116 may be configured to control the probe 106 during data acquisition. That is, the processor 116 may control the data acquisition by controlling which of the signal elements 108 are active and by controlling a shape of the beam emitted from the probe 106. For example, the processor 116 may be configured to control a transmit direction of the probe 106 such that data is acquired from a number of planes to generate a 3D scene.

Alternatively or additionally, the processor 116 may include a complex demodulator configured to demodulate radio frequency (RF) data obtained by the probe 106 and generate raw data. According to other embodiments, the demodulation of the RF data may be performed by another component of the ultrasound imaging system 100. The processor 116 may perform the processing operations described herein according to a plurality of selectable ultrasound modalities.

Depending on a mode of operation of the ultrasound imaging system 100, the processor 116 may process ultrasound data obtained by the probe 106 according to the mode of operation to generate image data. For example, the mode of operation may include B-mode, color flow Doppler mode, M-mode, color M-mode, spectral Doppler, elastography, TVI, strain, strain rate, and the like. Various of these modes of operation may be configured to, for instance, convert ultrasound data from beam space coordinates (e.g., received from the receive beamformer 112) to display space coordinates (e.g., such that the ultrasound data may be displayed as image data). In some embodiments, the mode of operation may allow for video processing by the processor 116 such that a series of images (e.g., processed ultrasound data) may be displayed in real-time while a scanning session/procedure is being performed on a patient. An operator of the ultrasound imaging system 100 (e.g., a sonographer) may switch between various modes to obtain a variety of ultrasound data and to perform a complete scan of an anatomical region of interest. For example, the operator may switch between modes using user interface 130 (e.g., using physical controls, interface inputs representing physical controls, etc.).

The processor 116 performs the processing operations in real-time as the echo signals are received by the receiver 110 from the probe 106. For the purposes of this disclosure, the term “real-time” is defined to include a procedure that is performed without any intentional delay. As an illustrative, non-limiting example, in certain instances, the ultrasound imaging system 100 may obtain images at a real-time volume-rate of 7-20 volumes/sec. It should be appreciated, however, that the real-time volume-rate may be dependent on the length of time that it takes to obtain each volume of data for display. Thus, the ultrasound imaging system 100 may be configured to obtain 2D data of an anatomical region at a faster rate than 3D data of the same anatomical region because it takes longer to obtain a volume of 3D data than the same volume of 2D data. Similarly, when the ultrasound imaging system 100 obtains a relatively large volume of data, the real-time volume-rate may be slower than for a smaller volume of data. For example, during an abdominal scan, the real-time volume-rate may be slower if the patient is an adult versus if the patient is an infant because the volume of data is larger for the adult than for the infant (e.g., due to the abdomen of an adult being larger than the abdomen of an infant). Therefore, certain implementations of the ultrasound imaging system 100 may have real-time volume-rates that are faster than 20 volumes/sec, while other implementations of the ultrasound imaging system 100 may have real-time volume-rates that are slower than 7 volumes/sec.

In some embodiments, the ultrasound imaging system 100 may include multiple processors configured to perform the processing operations/functionality described with reference to processor 116. For example, in such embodiments, a first processor of the multiple processors may be configured to demodulate and decimate the RF signal while a second processor of the multiple processors may be configured to further process the RF data prior to displaying an image representative of the data. It should be appreciated that other embodiments may use a different arrangement of processors.

The processor 116 may also be in electronic communication with the display device 132 such that the processor 116 may process ultrasound data obtained by the probe 106 and generate images to display on the display device 132.

As shown in FIG. 1, the processing circuit 114 also includes the memory 118. The memory 118 may be configured to, for example, store processed volumes of data obtained by the ultrasound imaging system 100 (e.g., ultrasound data collected by the probe 106). For example, the memory 118 may be a hospital picture archiving and communication system (PACS). The memory 118 (e.g., memory, memory unit, storage device, etc.) may include one or more devices (e.g., RAM, ROM, Flash memory, hard disk storage, etc.) for storing data and/or computer code for completing or facilitating the processes, layers, and modules described in the present application. The memory 118 may be or include tangible, non-transient volatile memory or non-volatile memory. The memory 118 may also include database components, object code components, script components, or any other type of information structure for supporting the activities and information structures described in the present application.

In various embodiments, the memory 118 may have varying capacity (e.g., storage space) across embodiments of the ultrasound imaging system 100. For example, the memory 118 may be configured to store at least 60 minutes’ worth of ultrasound data. The ultrasound data may be stored in the memory 118 such that the ultrasound data may be retrieved according to an order/time of acquiring the data. That is, the ultrasound data may be stored with a timestamp indicating a time at which the ultrasound data was collected and may be retrieved starting with an oldest time at which the ultrasound data was collected.

The processing circuit 114 also includes the image processing circuit 120, the AI circuit 122, and the control circuit 124. Each of the image processing circuit 120, the AI circuit 122, and the control circuit 124 are configured to facilitate determination of the second transmit direction using the probe 106 during an ultrasound scan.

The image processing circuit 120 is configured to analyze ultrasound image data (e.g., obtained using the probe 106, stored in the memory 118, etc.) and identify anatomical structures, scanning planes, transmit directions, pathologies, abnormalities, and/or other features depicted by/contained within the image data. In some instances, the image processing circuit 120 may include multiple deep learning-based models configured to analyze the image data. Alternatively or additionally, the image processing circuit 120 may use multiple deep learning-based models included in the AI circuit 122 to analyze the image data, as described herein.

In some embodiments, the image processing circuit 120 may be configured to identify an anatomical structure, feature, region, etc. captured by the image data. For example, during a laparoscopic ultrasound, the image processing circuit 120 may be configured to identify a liver, an abnormality, and/or other structures/features/regions depicted in the image data. The image processing circuit 120 may be configured to identify the anatomical structure using one or more algorithms (e.g., image processing algorithms such as edge detection, machine learning models, deep neural networks, etc.). In some embodiments, the image processing circuit 120 may be configured to apply one or more algorithms used by the AI circuit 122 and/or retrieved from the external database 128. For example, the image processing circuit 120 may use one or more algorithms to assess the liver 700 (e.g., using a segmentation model to segment the liver 700) captured by the image data. In this way, the image processing circuit 120 may be configured to estimate a location of the liver 700 and a location of an abnormality within the liver 700 (e.g., tumor 304) such that a resection volume based on the location of the liver 700 and the abnormality, as described herein. The ultrasound beam refers to the transmitted ultrasound signal. In another example, as described below, the image processing circuit 120 may use one or more algorithms to assess the abnormality captured/determined by the image data.

In some embodiments, the image processing circuit 120 may identify anatomical features such as bones, blood vessels, organs, etc., based on a shape, relative proximity, apparent depth, orientation, etc. of said features in the image data. Then, based on the identified anatomical features, the image processing circuit 120 may be configured to determine the anatomical structure depicted in the image data. For example, the image processing circuit 120 may determine that a liver is being imaged based on a movement and/or orientation of the liver relative to surrounding structures, such as blood vessels, via a deep learning classification model trained to recognize the movement and/or orientation of the liver, and other anatomical structures. In some embodiments, the image processing circuit 120 may detect movement of structures in the image data by comparing the location, shape, size, etc., of identified structures across a set of images (e.g., a cine loop).

The image processing circuit 120 may also be configured to determine the presence of a pathology (e.g., an injury, disease, abnormality, etc.) in the image data. In some embodiments, the image processing circuit 120 may use a deep learning classification model trained to recognize various pathologies in the anatomical structure represented by the image data to specify the pathology that is present. Continuing with the example of the laparoscopic ultrasound, the image processing circuit 120 may use a deep learning classification model trained to recognize pulmonary pathologies to determine whether a pathology is present in the image data from the laparoscopic ultrasound. For instance, the image processing circuit 120 may identify a tumor in the liver as a pathology present in the image data. In some embodiments, the AI circuit 122 may be configured to perform any of the functions of the image processing circuit 120 described herein using multiple deep learning-based models configured to analyze the image data.

Based on the analysis of the image data, processing circuit 114 (e.g., the AI circuit 122) may be configured to generate a resection volume. In some embodiments, the resection volume may be determined based on a position of the abnormality. For example, during a laparoscopic ultrasound, the image processing circuit 120 may generate a 3D scene and determine a position of a tumor of the liver (e.g., using a plurality of images obtained from an ultrasound signal), and the processing circuit 114 may determine, a resection volume to adjust to in which the planned resection volume does not interfere with another anatomical feature, such as a blood vessel. As another example, the control circuit 124 may determine that a planned resection volume interferes with another anatomical feature, prompting the need to adjust the resection volume. In this way, the image captured of the first anatomical feature (e.g., the liver 700) and the abnormality may be analyzed to determine a resection volume (e.g., resection volume 310) in which a second anatomical feature does not interfere with.

In some embodiments, the processing circuit 114 (e.g., the AI circuit 122) may be configured to automatically generate a control signal prompting an adjustment of the resection volume based on the analysis of the image data performed by the image processing circuit 120. The control signal may be received by the control circuit 124, which may be configured to automatically adjust the resection volume such that the planned resection volume does not interfere with a second anatomical feature.

The ultrasound imaging system 100 may also include an external database 128 and a user interface 130. The external database 128 refers to a database from which the processing circuit 114 (e.g., the image processing circuit 120, the AI circuit 122) retrieves information used in ultrasound imaging during a laparoscopic ultrasound. For example, the external database 128 may be a medical information database. The medical information database may store clinical guidelines, standard practices, medical literature, medical textbooks, published research, previous case studies, and so on. Depending on an implementation of the ultrasound imaging system 100 and/or a procedure performed thereby, the processing circuit 114 may retrieve clinical guidelines, standard practices, medical literature, medical textbooks, published research, and previous case studies related to the implementation and/or procedure. For example, if the ultrasound imaging system 100 is being used in a hospital setting to conduct a laparoscopic ultrasound, the processing circuit 114 may retrieve clinical guidelines and standard practices related to the hospital setting and the laparoscopic ultrasound. Continuing with this example, the processing circuit 114 may also retrieve information from the medical literature, medical textbooks, published research, and previous case studies related to pulmonary anatomy and the laparoscopic ultrasound. In some instances, the information retrieved from the external database 128 may include one or more algorithms used to segment the anatomical feature and determine a position of the anatomical feature.

The user interface 130 may be used by a sonographer or other clinician to control operation of the ultrasound imaging system 100. For example, the sonographer may use the user interface 130 to control the input of patient data, to change a scanning or display parameter, and/or to select various other modes, operations, parameters, etc. of the ultrasound imaging system 100. In some embodiments, the user interface 130 may be used to adjust the resection volume or adjust a determination of the resection volume. In some embodiments, the user interface 130 may include an off-the-shelf consumer electronic device such as a smartphone, a tablet, a laptop, and so on. For the purposes of this disclosure, the term “off-the-shelf consumer electronic device” is defined to be an electronic device that was designed and developed for general consumer use and one that was not specifically designed for use in a medical environment. Alternatively, in other embodiments, the user interface 130 may be an electronic device that was designed and developed for use in a medical environment.

According to some embodiments, the user interface 130 may be physically separate from the rest of the ultrasound imaging system 100 (e.g., the transmit beamformer 102, the transmitter 104, the probe 106, the receiver 110, the receive beamformer 112, the processing circuit 114, and/or the external database 128). The user interface 130 may communicate with the processor 116 through a wireless protocol, such as Wi-Fi, Bluetooth, wireless local area network (WLAN), near-field communication, and so on. According to some embodiments, the user interface 130 may communicate with the processor 116 through an application programming interface (API).

In some embodiments, the user interface 130 may include physical controls such as one or more of buttons, sliders, a rotary knob, a mouse, a keyboard, a trackball, hard keys linked to specific actions, soft keys that may be configured to control different functions, and so on. As shown in FIG. 1, the user interface 130 may also include a display device 132. In some embodiments, the display device 132 may be configured to display a graphical user interface (GUI) based on an instruction from the memory 118. The GUI may include user interface icons representing commands and instructions relating to the operation of the ultrasound imaging system 100. The user interface icons of the GUI may be configured such that a user (e.g., the sonographer, clinician, etc.) may select a specific user interface icon to initiate a specific function controlled by the GUI. For example, various user interface icons may be used to represent windows, menus, buttons, cursors, scroll bars, and so on. That is, the physical controls of the user interface 130 may be included as individual hardware elements, as user interface icons displayed on the display device 132, or as a combination of hardware elements and user interface icons.

In some embodiments, the display device 132 may include a touch-sensitive display device or a touch screen. According to such embodiments, the touch screen may be configured to interact with the GUI displayed by the display device 132 such that a user (e.g., the sonographer) can interact with the GUI via the touch screen. The touch screen may be a single-point touch screen that is configured to detect a single contact point at a time, or the touch screen may be a multi-point touch screen that is configured to detect multiple points of contact at a time. For embodiments where the touch screen is a multi-point touch screen, the touch screen may be configured to detect multi-point gestures involving contact from two or more of a user's fingers at a time. The touch screen may be a resistive touch screen, a capacitive touch screen, or any other type of touch screen that is configured to receive inputs from a stylus or one or more of a user's fingers. According to some embodiments, the touch screen may be an optical touch screen that uses technology such as infrared light or other frequencies of light to detect one or more points of contact initiated by a user. In some embodiments, the touch screen may be incorporated as part of the display device 132 or may be separate from the display device 132. The user interface 130 may also include a proximity sensor configured to detect objects and/or gestures that are within a predetermined distance (e.g., five feet, six inches, ten centimeters, etc.) of the proximity sensor. In various embodiments, the proximity sensor may be located on the display device 132 or as part of a touch screen that is separate from the display device 132.

Referring to FIG. 2, an illustration of a scan volume 202 is shown. The scan volume 202 includes a plurality of planes 204. The plurality of planes 204 are taken along different location to form the scan volume 202. In various embodiments, the scan volume 202 is formed using a two-dimensional probe 106. The probe 106 along with a spatial tracking sensor (e.g., a position sensor, etc.) can be used to acquire the plurality of planes 204. The planes 204 are then combines to create the scan volume 202. The scan volume 202 creates a 3D scene (e.g., a 3D model). In various embodiments, a general region of an anatomical feature (e.g., liver 700) and/or an abnormality (e.g., tumor 304) is known to decide where to initially position the probe 106 for scanning. The spatial tracking sensor uses positional information to aid in building the scan volume 202 with the planes 204.

Advantageously, the scan volume 202, and the 3D scene, can be formed without reliance on an operator’s expertise/skills. When capturing images and information to create a 3D scene, visual information of the probe 106 position may be limited. The usage of the spatial tracking sensor for position information to build the 3D scene allows for the 3D scene to be efficiently built.

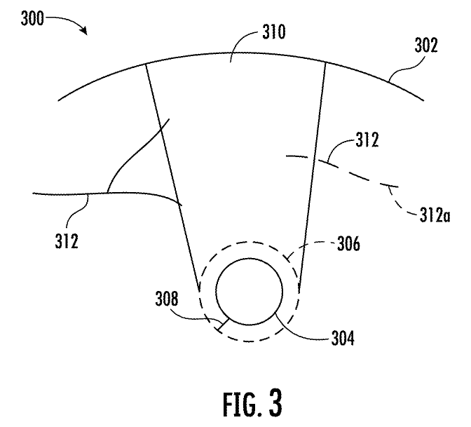

Referring to FIG. 3, an illustration of a first anatomical feature 300 (e.g., liver 700) is shown. The first anatomical feature 300 includes a surface 302 of the first anatomical feature 300. The surface 302 can be identified based on a 3D scene created (e.g., as described with reference to the scan volume 202). Although shown in FIG. 3 along a two-dimensional space, the 3D scene is along a three-dimensional space, such that depth and extension information is captured from the 3D scene. In various embodiments, the surface 302 is identified based on a position of the probe 106 as the first anatomical feature 300 is scanned such that the probe 106 follows the surface 302 during scanning. In other words, as a translational sweep of a region (e.g., a region including tumor 304) can allow for the surface 302 to be identified and the 3D scene to be created. The first anatomical feature 300 includes an abnormality, shown as a tumor 304. The tumor 304 may be located with the usage of ultrasound imaging. For example, as described herein with reference to step 410, step 510, and step 610, various embodiments of ultrasound imaging can be used to determine a position of the tumor 304. For example, the image processing circuit 120 can be used to determine a position of the tumor 304. In some embodiments, the tumor 304 is positioned (automatically or manually) based on fusing the 3D scene created with pre-operative data.

Using information on the tumor 304 and patient, a tumor margin 306 is selected. The tumor margin 306 is selected to ensure resection of the tumor 304 removes as much abnormal (e.g., cancerous) cells. A margin size 308 of the tumor margin 306 may be dependent on the type of cancer the tumor 304 is associated with and/or surgical guidelines. For example, the margin size 308 may be between 1 to 2 millimeters for a tumor 304 associated with liver cancer. In various embodiments, there is not tumor margin 306.

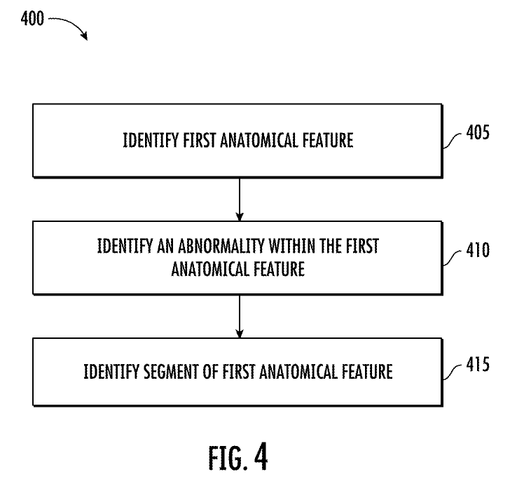

With the determination of the surface 302, the tumor 304, and the tumor margin 306, a resection volume 310 can be determined. In various embodiments, the 310 is a conic shape. The resection volume 310 may be based on a projection of the tumor 304 and the tumor margin 306 on the surface 302 of the first anatomical feature 300. Traditionally, as described with reference to FIG. 4, the resection volume 310 may be determined based on a segment of the first anatomical feature 300 that the tumor 304 is positioned in. The choice of the resection volume 310 affects how much tissue is being removed during resection. Determining how much tissue is being removed during a resection is indicative of the outcome of the procedure. If a resection volume 310 is initially determined, an operator, may want to update the resection volume 310 or a surgical strategy to minimize the amount of tissue to be removed.

As shown in FIG. 3, within the first anatomical feature 300 are a second anatomical feature 312. The second anatomical feature 312 interfere with the resection volume 310. In other words, at least a portion of the second anatomical feature 312 is within the resection volume 310. Ultrasound imaging can help in locating the resection volume 310 to identify second anatomical features 312 in proximity to the tumor 304 and the resection volume 310. However, as described herein, visual information from ultrasound imaging may be limited and reconstruction of the 3D scene may not provide enough information to accurately determine the second anatomical feature 312. The systems and methods described herein generate a 3D scene using two-dimensional imaging. The generated 3D scene can be used to identify second anatomical feature 312 in the resection volume 310. For example, the second anatomical feature 312 may be in a position 312a, as shown in FIG. 3, that interferes with the resection volume 310 but is not visible from a generated ultrasound image of the first anatomical feature 300. Using information from the generated 3D scene, it may be more efficient to identify second anatomical features 312 that interfere with the resection volume 310 (e.g., by the processing circuit 114, the control circuit 124). With this information, the resection volume 310 can be adapted. In various embodiments, as shown in FIG. 3, the second anatomical feature 312 is a blood vessel. The second anatomical feature 312 may be a major blood vessel, such that a user is notified to adapt the resection volume 310 to avoid the major blood vessel. The second anatomical feature 312 may be a blood vessel that is not major, such that an operator may not be notified to adapt the second anatomical feature 312. Calculating the resection volume 310 and displaying whether second anatomical features 312 interfere with the resection volume 310, as described herein, provides an operator with more information during resection planning to reduce healthy tissues being removed and reduce the likelihood of failure during surgery.

Referring to FIG. 4, a flow chart illustrating a method 400 for anatomical resection planning is shown. During anatomical resection, as described herein, a plurality of healthy tissue is removed due to the method. This may result in postoperative failure of the organ.

As shown in FIG. 4, at step 405, method 400 includes identifying a first anatomical feature. An ultrasound imaging system with a transducer configured to transmit and receive an ultrasound may be used to identify the first anatomical feature.

As shown in FIG. 4, method 400 includes identifying an abnormality within the first anatomical feature at step 410. In various embodiments, the ultrasound imaging system as described with reference to step 405 is used to identify the abnormality. The abnormality may be a tumor. In various embodiments, step 410 may also include identifying second anatomical features in proximity to the abnormality.

Method 400 includes identifying a segment of the first anatomical feature at step 415. The segment of the first anatomical feature includes the abnormality. With an anatomical resection, anatomical segments of the first anatomical feature are resected rather than an area just of the abnormality. Due to the resection of anatomical segments with anatomical resections, more healthy tissues are being removed than with non-anatomical resections. As such, it is advantageous to move towards non-anatomical resections, as described with the method 500 and method 600 to remove less healthy tissues to maintain function of the first anatomical feature.

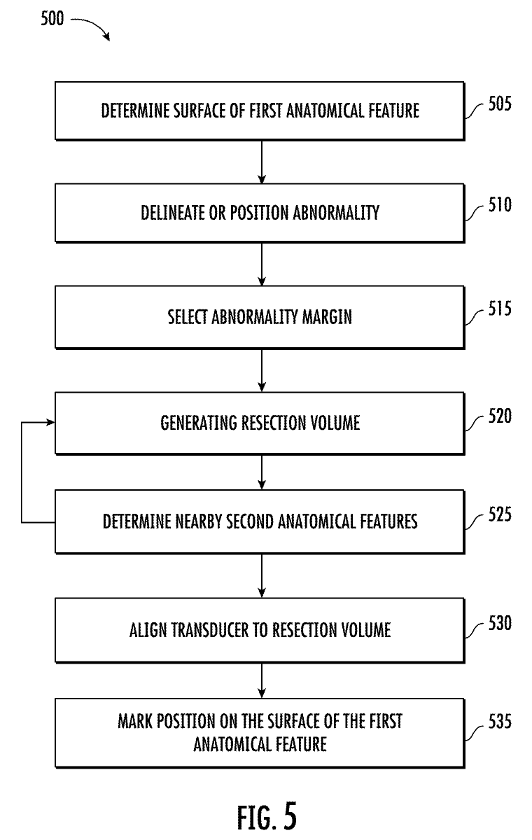

Referring to FIG. 5, a flow chart illustrating a method 500 for non-anatomical resection planning is shown. Method 500 utilizes an ultrasound imaging system. In at least one embodiment, the ultrasound imaging system referred to by method 500 is the ultrasound imaging system 100 described above with reference to FIG. 1, and method 500 may be implemented by the ultrasound imaging system 100. In some embodiments, method 500 may be implemented as executable instructions in a memory of the ultrasound imaging system 100, such as the memory 118 of FIG. 1.

As shown in FIG. 5, at step 505, method 500 includes determining a surface of a first anatomical feature. The surface of the first anatomical feature may be determined with the ultrasound imaging system 100 as described with reference to surface 302 of the first anatomical feature 300 in FIG. 3. For example, the transducer of the ultrasound imaging system 100 is configured to transmit and receive an ultrasound signal. The ultrasound signal can be used to generate a 3D scene that includes the first anatomical feature. Using the 3D scene, the surface of the first anatomical feature can be determined. Step 505 may include processing of the ultrasound signal to generate the 3D scene. A plurality of images along several planes (e.g., planes 204) is sampled with the usage of the probe 106 and a position sensor. Processing of the images with the position sensor data allows for the generation of the 3D scene.

Method 500 includes delineating or positioning an abnormality of the first anatomical feature at step 510. The abnormality can be positioned within the 3D scene based on the ultrasound signal from step 505. In various embodiments, the position of the abnormality within the 3D scene is determined using preoperative data. For example, a previous scan of the first anatomical feature may be using to determine the position of the abnormality. The abnormality may be automatically positioned using preoperative data and/or the ultrasound signal. In various embodiments, the operator may manually position the abnormality within the 3D scene.

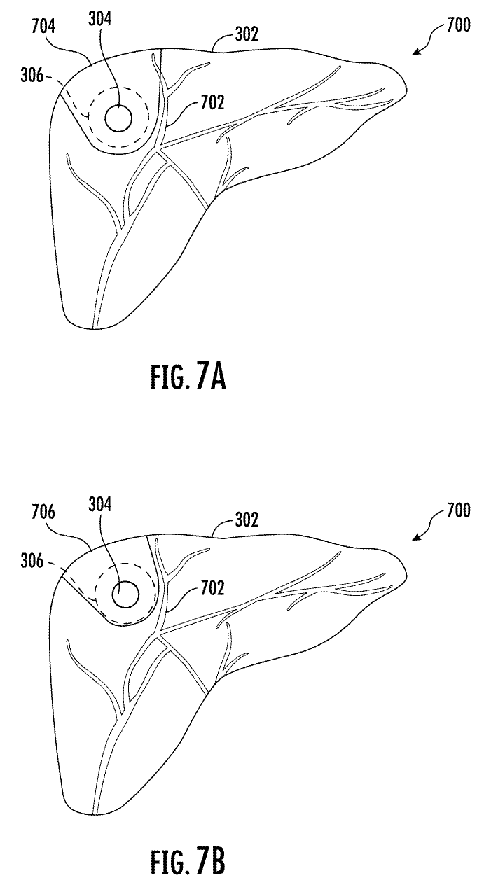

Method 500 includes selecting an abnormality margin at step 515. As described herein with reference to the tumor margin 306, the abnormality margin is selected to ensure proper resection of the abnormality and may be dependent on the type of pathology the abnormality is associated with and/or surgical guidelines. The abnormality margin may be set by the operator such that the processing circuit 114 can receive the abnormality margin and set the abnormality margin within the 3D scene, as shown in FIGS. 7A and 7B, among others.

Method 500 includes generating a resection volume at step 520. The resection volume is a section of the first anatomical feature that includes the abnormality and the abnormality margin. Contrary to step 415 of method 400, the resection volume at step 520 is generated based on the abnormality and the abnormality margin rather than being an anatomical section of the first anatomical feature which is the section at step 415. The resection volume at step 520 is generated based on the surface of the first anatomical feature, the abnormality, and the abnormality margin. In various embodiments, the resection volume is conic shape or frustoconical. The resection volume may be a projection of the abnormality and the abnormality margin on the surface of first anatomical feature.

As shown in FIG. 5, method 500 includes determining nearby second anatomical features at step 525. The second anatomical features can be determined using the 3D scene generated. The processing circuit 114 may be configured to identify and determine the second anatomical features. As described herein, the second anatomical features may be vessels. At a determination that the second anatomical feature interferes with the resection volume, method 500 may return to step 520 to adapt the resection volume. Adaptation of the resection volume may include changing a direction and/or angle of the projection to determine the adapted resection volume. In various embodiments, an operator may manually adapt the projection to change the resection volume. In various embodiments, the resection volume is automatically updated based on the identified second anatomical features.

In various embodiments, at step 525, the processing circuit 114 may determine the second anatomical feature interferes with the resection volume and determine that the resection volume does not need to be adapted based on the second anatomical feature not meeting a threshold. For example, if the second anatomical feature is not a major blood vessel (e.g., a blood vessel with a width greater than 3 millimeters), the processing circuit 114 may determine that the resection volume does not need to be adapted.

Method 500 includes aligning the transducer to the resection volume at step 530. Based on the adapted resection volume, the transducer can be aligned in the direction of the resection volume. Step 530 may include an operator manually adjusting the probe 106 to align the transducer. Aligning the transducer at step 530 allows for a position on the first anatomical feature to be marked at step 535. The marked position at step 535 allows the operator to know where the resection should be located. Advantageously, the method 500 with the ultrasound imaging system 100 as described herein allows for a resection volume that minimizes interference with second anatomical features to be determined and marked. As a result, intraoperative and postoperative failures can be reduced. For example, in the embodiment where the second anatomical feature is a blood vessel, by adaptation of the resection volume to not interfere with the blood vessel. a risk of bleeding, which is a common complication with non-anatomical resection, is minimized.

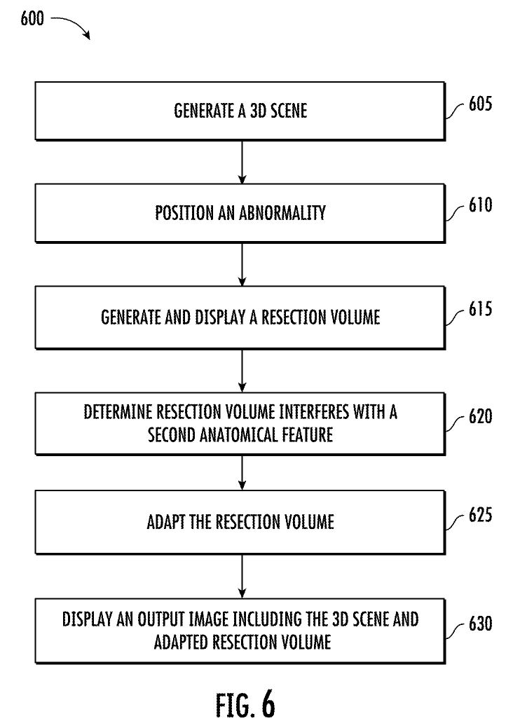

Referring to FIG. 6, a flow chart illustrating a method 600 for ultrasound imaging using an ultrasound imaging system is shown. In at least one embodiment, the ultrasound imaging system referred to by method 600 is the ultrasound imaging system 100 described above with reference to FIG. 1, and method 600 may be implemented by the ultrasound imaging system 100. In some embodiments, method 600 may be implemented as executable instructions in a memory of the ultrasound imaging system 100, such as the memory 118 of FIG. 1.

As shown in FIG. 6, method 600 includes generating a 3D scene at step 605. The 3D scene includes a first anatomical feature. As described herein, the transducer of the ultrasound imaging system 100 is configured to transmit and receive an ultrasound signal. Based on the ultrasound signal, the 3D scene is generated. For example. as described with reference to FIG. 2, the probe 106 and a position sensor may be used to scan an area including the first anatomical feature to obtain a plurality of image data in a number of planes (e.g., plurality of planes 204). Using position information from the position sensor, the 3D scene may be generated. Processing of the ultrasound signals allows for image construction. Processing ultrasound signals may include converting the received echoes from the probe 106 into electrical signals, passing the electrical signals through the receive beamformer 112 for ultrasound data, and performing beamforming techniques to generate an image. In various embodiments, the image processing circuit 120 applies processing techniques to the constructed image to further improve quality of the constructed images (e.g., contrast adjustment, brightness adjustment, noise reduction, etc.). In some embodiments, step 605 may be performed by the processing circuit 114. For example. as described herein image data may be obtained and the image processing circuit 120 may be configured to generate the 3D scene based on the ultrasound signal. In various embodiments, the first anatomical feature is a liver. For example, the imaging of the liver may occur during a laparoscopic ultrasound.

Generating the 3D scene including the first anatomical feature at step 605 may include segmenting the first anatomical feature. Segmentation of the first anatomical feature can produce a binary mask of the first anatomical feature. Although various methods can be employed to segment the first anatomical feature, one way to do so is by training a segmentation model (e.g., U-Net, Mask R-CNN, DeepLab, etc.). The trained model can analyze an image data taken along the second planes and output a probability for each data point (e.g., pixel) of the image data being the first anatomical feature or a background, i.e., not the first anatomical feature. Setting a threshold for the probabilities to determined which pixels classify as the first anatomical feature then creates the binary mask of the first anatomical feature for identification. Segmentation of the first anatomical feature allows for a location of the first anatomical feature to be estimated. As described herein, by determining the location of the first anatomical feature, a position of an abnormality within the first anatomical information and a resection volume can be determined.

Method 600 includes positioning of an abnormality of the first anatomical feature at step 610. The abnormality is positioned in the 3D scene. In various embodiments, the abnormality is a tumor. Continuing the example in which the first anatomical feature is the liver, the laparoscopic ultrasound imaging may be used for a patient with liver cancer. As described with reference to step 510 of method 500, the position of the abnormality may be determined based on the ultrasound signal. For example, ultrasound images received along with ultrasound frame tracking may be used to position the abnormality within the 3D scene. Ultrasound frame tracking can be aided by the AI circuit 122 and the position sensor, among others. In other embodiments, the position of the abnormality may be determined based on preoperative data. For example, previous ultrasound images may be used to determine and position the abnormality within the 3D scene. In various embodiments, ultrasound imaging may not be used to determine the position of the abnormality. For example, the abnormality may not be visible in an ultrasound scan. A different imaging modality (e.g., computed tomography, magnetic resonance imaging, etc.) may be used to determine the position of the abnormality. The abnormality may then be positioned in the 3D scene through registration between the different imaged modality and the ultrasound images. Registration can be done manually, automatically, with machine learning, etc. In some embodiments, step 610 may be performed by the processing circuit 114. For example, the image processing circuit 120 may be configured to determine the position of the abnormality.

Method 600 includes generating and displaying a resection volume at step 615. The resection volume is generated on the first anatomical feature within the 3D scene based on the position of the abnormality. A position and shape of the resection volume may be based on a projection of the abnormality on a surface of the first anatomical feature. In various embodiments, the shape of the resection volume is conical or frustoconical. Step 615 may be performed by the processing circuit 114. For example, the control circuit 124 can be configured to generate and display the resection volume on the first anatomical feature within the 3D scene. In various embodiments, the generated and displayed resection volume is a user input. For example, an operator may determine the resection volume by adjusting the user interface 130. The 3D scene including the resection volume can be displayed on the display device 132 of the ultrasound imaging system 100, allowing the operator to view the resection volume. In various embodiments, the user interface 130 of the ultrasound imaging system 100 allows for the operator to adjust a view of the 3D scene and highlight specific portions of the 3D scene. For example, the resection volume and/or the abnormality may be highlighted.

In various embodiments, as described with reference to step 515 of method 500, method 600 may include receiving an abnormality margin. The abnormality margin may be received based on the abnormality. In various embodiments, the abnormality margin may be received by the processing circuit 114 (e.g., the control circuit 124) and the external database 128. For example, the processing circuit 114 may receive a user input based on the abnormality margin. The resection volume generated at step 615 may then be determined based on the first anatomical feature, the abnormality, and the abnormality margin. For example, as shown in FIGS. 3, 7A and 7B, the resection volume can be a projection of the abnormality and the abnormality margin on the surface of the first anatomical feature.

Method 600 includes determining the resection volume interferes with a second anatomical feature at step 620. The determination can be made based on data from the 3D scene and the resection volume generated at step 615. Step 602 may be performed by the processing circuit 114. In various embodiments, the image processing circuit 120 is configured to identify the second anatomical feature and the control circuit 124 may use position data of the identified anatomical feature to determine the resection volume interferes with the second anatomical feature.

Identifying the second anatomical feature at step 620 may include segmenting the segmenting anatomical feature. As described herein with reference to segmenting the first anatomical feature at step 605, segmentation of the second anatomical feature can produce a binary mask of the second anatomical feature. Segmentation of the second anatomical feature allows for a location of the second anatomical feature to be estimated. As described herein, determining the location of the second anatomical feature aids in determining if the second anatomical feature interferes with the resection volume and in determining if adaptation of the resection volume is needed.

Step 620 may also include determining the resection volume is within a margin of the second anatomical feature. For example, determining the resection volume interferes with the second anatomical feature may be based on determining the resection volume is within the margin. The resection volume may not interfere with the second anatomical feature but may be within a margin of the anatomical feature, such that the resection volume should be adjusted.

In various embodiments, the second anatomical feature is a blood vessel. As described with reference to step 525 of method 500, the blood vessel may be a major blood vessel (e.g., a blood vessel having a width greater than 3 millimeters (mm)). The processing circuit 114 may be configured to identify and determine the resection volume interferes with the second anatomical feature for only major blood vessels and/or not all blood vessels.

The displayed 3D scene including the resection volume may include the second anatomical feature interfering with the resection volume highlighted. As a result, the operator can clearly view the second anatomical features during resection planning. In various embodiments, the user interface 130 may allow for customization during resection planning. The operator may be able to adjust which anatomical features should be marked as the second anatomical feature (e.g., major blood vessels). For example, the operator may be able to define a width range (e.g., greater than 3 mm) of blood vessels that should be marked as the second anatomical feature for determining interference with the resection volume. The operator may also use the user interface 130 to adjust the margin of the second anatomical feature to the resection volume for determining the interference.

Method 600 includes adapting the resection volume at step 625. The resection volume is adapted based on the determination that the planned resection volume at interferes with the second anatomical feature at step 620. As described herein, the resection volume may be adapted by adjusting the projection of the abnormality on the surface of the first anatomical feature. For example, an angle of the projection relative to the surface and the abnormality may be adjusted to adapt the resection volume. Step 625 can be performed by the processing circuit 114. For example, the control circuit 124 may be configured to adapt the resection volume. In various embodiments, the abnormality margin may be adjusted to adapt the resection volume. For example, the abnormality margin can be reduced to avoid the resection volume interfering with the second anatomical feature. The operator may input a user input on the user interface 130 to adjust the abnormality margin. The processing circuit 114 (e.g., the control circuit 124) can receive the user input to adjust the abnormality margin and as a result, the resection volume is adapted based on the user input. The resection volume can be manually adapted by the operator. For example, the operator may adjust the probe 106 and/or the user interface 130 to adapt the resection volume. The resection volume can be automatically adapted. For example, the resection volume can be automatically adapted based on a location of the first anatomical feature and the second anatomical feature. Using the determined location of the second anatomical feature, an adapted resection volume that does not interfere the second anatomical feature can be determined.

In various embodiment, the resection volume is adapted at step 625 based on a physical change of the first anatomical feature. As described herein, resection volumes are generally planned using preoperative data. Preoperative data may be acquired a substantial time ago, to which the first anatomical feature may have deformed or changed by then. The systems and methods described herein allow for the resection volume to be determined based on a current anatomy of the first anatomical feature, which results in a more accurate determination of the resection volume. The physical change of the first anatomical feature can be taken into account when the determination and adaptation of the resection volume occurs. Using the physical change of the first anatomical feature (i.e., the current anatomy of the first anatomical feature), the processing circuit 114 (e.g., the control circuit 124) can update the determination that the resection volume interferes with the second anatomical feature and adapt the resection volume if necessary.

As shown in FIG. 6, method 600 includes displaying an output image including the 3D scene and the adapted resection volume at step 630. In some embodiments, the output image is displayed on the display device 132. The output image may include the 3D scene and/or multiple image data extracted from the 3D scene, such that an operator can visualize the adapted resection volume and the second anatomical features. In various embodiments, the operator can manipulate the 3D scene using the user interface 130.

Referring to FIGS. 7A and 7B, images of a first anatomical feature are shown. The first anatomical feature shown is a liver 700. A 3D scene including the liver 700 is generated using method 600 (e.g., step 605) as described herein. The liver 700 includes a tumor 304. A position of the tumor 304 can be determined with preoperative data, such that the tumor 304 may be positioned within the liver 700 in the 3D scene. As shown in FIG. 7A, a resection volume 704 is generated based on the tumor 304 and a tumor margin 306 of the tumor 304. The resection volume is a projection of the tumor 304 and the tumor margin 306 on a surface 302 of the liver 700. As shown in FIG. 7A, the planned resection volume 704 interferes with a blood vessel 702. The interference of the planned resection volume 704 with the blood vessel 702 may not have been determined in initial preoperative data. However, as the liver 700 deforms, the planned resection volume 704 may be shown to interfere with the blood vessel 702, highlighting an advantage of the systems and methods described herein which allows for an estimate of the resection volume based on a current anatomy of the liver 700. With the determination that the planned resection volume 704 interferes with the blood vessel 702, the planned resection volume 704 may be adapted to generate an adapted resection volume 706, shown in FIG. 7B. The adapted resection volume 706 does not intersect with the blood vessel 702. As shown in FIG. 7B, an angle of the projection of the tumor 304 and the tumor margin 306 on the surface 302 is adjusted to generate the adapted resection volume 706.

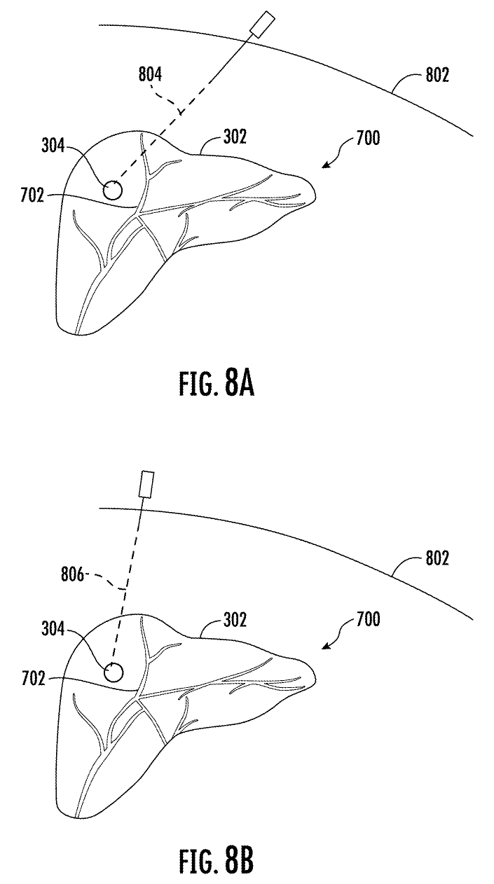

The systems and methods described herein may also be applied to needle ablation. Rather than generation and adaptation of a resection volume, a needle ablation can be generated and adapted. Referring to FIGS. 8A and 8B, images of a first anatomical feature are shown. The first anatomical feature shown is a liver 700. A 3D scene including the liver 700 is generated using method 600 (e.g., step 605) as described herein. The liver 700 includes a tumor 304. A position of the tumor 304 can be determined with preoperative data, such that the tumor 304 may be positioned within the liver 700 in the 3D scene. As shown in FIG. 8A, a needle ablation 804 is generated based on the tumor 304. The needle ablation 804 is planned to go through a skin surface 802 of the patient and through to the tumor 304. As shown in FIG. 8A, the planned needle ablation 804 interferes with a blood vessel 702. With the determination that the planned needle ablation 804 interferes with the blood vessel 702, the planned needle ablation 804 may be adapted to generate an adapted needle ablation 806, shown in FIG. 8B. The adapted needle ablation 806 does not intersect with the blood vessel 702. As shown in FIG. 8B, an angle of the planned needle ablation 804 is adjusted to generate the adapted needle ablation 806. In various embodiments, this adjustment may occur automatically (e.g., by the processing circuit 114)

The embodiments described herein have been described with reference to drawings. The drawings illustrate certain details of specific embodiments that provide the systems, methods and programs described herein. However, describing the embodiments with drawings should not be construed as imposing on the disclosure any limitations that may be present in the drawings.

It should be understood that no claim element herein is to be construed under the provisions of 35 U.S.C. § 112(f), unless the element is expressly recited using the phrase “means for.”

As utilized herein, terms of degree such as “approximately,” “about,” “substantially,” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to any precise numerical ranges provided. Accordingly, these terms should be interpreted as indicating that insubstantial or inconsequential modifications or alterations of the subject matter described and claimed are considered to be within the scope of the disclosure as recited in the appended claims.

It should be noted that terms such as “exemplary,” “example,” and similar terms, as used herein to describe various embodiments, are intended to indicate that such embodiments are possible examples, representations, or illustrations of possible embodiments, and such terms are not intended to connote that such embodiments are necessarily extraordinary or superlative examples.

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

The term “or,” as used herein, is used in its inclusive sense (and not in its exclusive sense) so that when used to connect a list of elements, the term “or” means one, some, or all of the elements in the list. Conjunctive language such as the phrase “at least one of X, Y, and Z,” unless specifically stated otherwise, is understood to convey that an element may be either X, Y, Z; X and Y; X and Z; Y and Z; or X, Y, and Z (i.e., any element on its own or any combination of X, Y, and Z). Thus, such conjunctive language is not generally intended to imply that certain embodiments require at least one of X, at least one of Y, and at least one of Z to each be present, unless otherwise indicated.

References herein to the positions of elements (e.g., “top,” “bottom,” “above,” “below”) are merely used to describe the orientation of various elements in the drawings. It should be noted that the orientation of various elements may differ according to other exemplary embodiments, and that such variations are intended to be encompassed by the present disclosure.

As used herein, terms such as “engine” or “circuit” may include hardware and machine-readable media storing instructions thereon for configuring the hardware to execute the functions described herein. The engine or circuit may be embodied as one or more circuitry components including, but not limited to, processing circuitry, network interfaces, peripheral devices, input devices, output devices, sensors, etc. In some embodiments, the engine or circuit may take the form of one or more analog circuits, electronic circuits (e.g., integrated circuits (IC), discrete circuits, system on a chip (SOCs) circuits, etc.), telecommunication circuits, hybrid circuits, and any other type of circuit. In this regard, the engine or circuit may include any type of component for accomplishing or facilitating achievement of the operations described herein. For example, an engine or circuit as described herein may include one or more transistors, logic gates (e.g., NAND, AND, NOR, OR, XOR, NOT, XNOR, etc.), resistors, multiplexers, registers, capacitors, inductors, diodes, wiring, and so on).

An engine or circuit may be embodied as one or more processing circuits comprising one or more processors communicatively coupled to one or more memory or memory devices. In this regard, the one or more processors may execute instructions stored in the memory or may execute instructions otherwise accessible to the one or more processors. The one or more processors may be constructed in a manner sufficient to perform at least the operations described herein. In some embodiments, the one or more processors may be shared by multiple engines or circuits (e.g., engine A and engine B, or circuit A and circuit B, may comprise or otherwise share the same processor which, in some example embodiments, may execute instructions stored, or otherwise accessed, via different areas of memory).

Alternatively or additionally, the one or more processors may be structured to perform or otherwise execute certain operations independent of one or more co-processors. In other example embodiments, two or more processors may be coupled via a bus to enable independent, parallel, pipelined, or multi-threaded instruction execution. Each processor may be provided as one or more suitable processors, application specific integrated circuits (ASICs), field programmable gate arrays (FPGAs), digital signal processors (DSPs), or other suitable electronic data processing components structured to execute instructions provided by memory. The one or more processors may take the form of a single core processor, multi-core processor (e.g., a dual core processor, triple core processor, quad core processor, etc.), microprocessor, etc. In some embodiments, the one or more processors may be external to the apparatus, for example the one or more processors may be a remote processor (e.g., a cloud based processor). Alternatively or additionally, the one or more processors may be internal and/or local to the apparatus. In this regard, a given engine or circuit or components thereof may be disposed locally (e.g., as part of a local server, a local computing system, etc.) or remotely (e.g., as part of a remote server such as a cloud based server). To that end, engines or circuits as described herein may include components that are distributed across one or more locations.

An example system for providing the overall system or portions of the embodiments described herein might include one or more computers, including a processing unit, a system memory, and a system bus that couples various system components including the system memory to the processing unit. Each memory device may include non-transient volatile storage media, non-volatile storage media, non-transitory storage media (e.g., one or more volatile and/or non-volatile memories), etc. In some embodiments, the non-volatile media may take the form of ROM, flash memory (e.g., flash memory such as NAND, 3D NAND, NOR, 3D NOR, etc.), EEPROM, MRAM, magnetic storage, hard discs, optical discs, etc. In other embodiments, the volatile storage media may take the form of RAM, TRAM, ZRAM, etc. Combinations of the above are also included within the scope of machine-readable media. In this regard, machine-executable instructions comprise, for example, instructions and data which cause a general purpose computer, special purpose computer, or special purpose processing machines to perform a certain function or group of functions. Each respective memory device may be operable to maintain or otherwise store information relating to the operations performed by one or more associated circuits, including processor instructions and related data (e.g., database components, object code components, script components, etc.), in accordance with the example embodiments described herein.

Although the drawings may show and the description may describe a specific order and composition of method steps, the order of such steps may differ from what is depicted and described. For example, two or more steps may be performed concurrently or with partial concurrence. Also, some method steps that are performed as discrete steps may be combined, steps being performed as a combined step may be separated into discrete steps, the sequence of certain processes may be reversed or otherwise varied, and the nature or number of discrete processes may be altered or varied. The order or sequence of any element or apparatus may be varied or substituted according to alternative embodiments. Accordingly, all such modifications are intended to be included within the scope of the present disclosure as defined in the appended claims. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

The foregoing description of embodiments has been presented for purposes of illustration and description. It is not intended to be exhaustive or to limit the disclosure to the precise form disclosed, and modifications and variations are possible in light of the above teachings or may be acquired from this disclosure. The embodiments were chosen and described to explain the principals of the disclosure and its practical application to enable one skilled in the art to utilize the various embodiments and with various modifications as are suited to the particular use contemplated. Other substitutions, modifications, changes and omissions may be made in the design, operating conditions, and arrangement of the embodiments without departing from the scope of the present disclosure as expressed in the appended claims.

What is claimed is:

1. An ultrasound imaging system comprising:
a transducer configured to transmit a transmit signal and receive an ultrasound signal; and
a processing circuit having a processor coupled to a memory device storing instructions thereon that, when executed, cause the processing circuit to perform operations comprising:
generating a three-dimensional (3D) scene based on the ultrasound signal including a first anatomical feature;
positioning an abnormality of the first anatomical feature in the 3D scene based on the ultrasound signal;
generating and displaying a resection volume on the first anatomical feature within the 3D scene based on the position of the abnormality;
determining the resection volume interferes with a second anatomical feature;
adapting the resection volume based on the determination that the planned resection volume interferes with the second anatomical feature; and
displaying an output image on a display screen of the ultrasound imaging system, the output image comprising the 3D scene including the adapted planned resection volume.

2. The ultrasound imaging system of claim 1, wherein the first anatomical feature is a liver and the second anatomical feature is a blood vessel.

3. The ultrasound imaging system of claim 2, wherein the blood vessel is a major blood vessel, the major blood vessel having a width greater than 3 millimeters.

4. The ultrasound imaging system of claim 1, wherein the operations further comprise receiving an abnormality margin and the resection volume is based on the first anatomical feature, the abnormality, and the abnormality margin.

5. The ultrasound imaging system of claim 4, wherein the operations further comprise receiving a user input to adjust the abnormality margin, and wherein adapting the resection volume is based on the user input.

6. The ultrasound imaging system of claim 1, wherein the position and shape of the resection volume is based on a projection of the abnormality on a surface of the first anatomical feature.

7. The ultrasound imaging system of claim 1, wherein determining that the planned resection volume interferes with the second anatomical feature is based on determining that the resection volume is within a margin of the second anatomical feature.

8. The ultrasound imaging system of claim 1, wherein adapting the resection volume is based on a physical change of the first anatomical feature.

9. The ultrasound imaging system of claim 1, wherein the displayed planned resection volume includes the second anatomical feature interfering with the resection volume highlighted.

10. The ultrasound imaging system of claim 1, wherein the resection volume is automatically adapted based on a location of the first anatomical feature and the second anatomical feature.

11. An ultrasound imaging system comprising:
a transducer configured to transmit a transmit signal and receive an ultrasound signal;
an image processing circuit configured to generate a 3D scene based on the ultrasound signal including a first anatomical feature and position an abnormality of the first anatomical feature; and
a control circuit configured to generate and display a resection volume on the first anatomical feature within the 3D scene based on the position of the abnormality and upon determining the planned resection volume interferes with a second anatomical feature, adapting the resection volume, such that the ultrasound imaging system is configured to display an output image on a display screen of the ultrasound imaging system, the output image comprising the 3D scene including the adapted planned resection volume.

12. The ultrasound imaging system of claim 11, wherein the first anatomical feature is a liver and the second anatomical feature is a blood vessel.

13. The ultrasound imaging system of claim 11, wherein the position and shape of the resection volume is based on a projection of the abnormality on a surface of the first anatomical feature.

14. The ultrasound imaging system of claim 11, wherein adapting the resection volume is based on a physical change of the first anatomical feature, the control circuit to update the determination that the resection volume interferes with the second anatomical feature as the first anatomical feature changes.

15. The ultrasound imaging system of claim 11, wherein the control circuit is further configured to:
receive an abnormality margin; and
receive a user input to adjust the abnormality margin, wherein the resection volume is based on the first anatomical feature, the abnormality, and the abnormality margin and adapting the resection volume is based on the user input.

16. A method comprising:
generating, by a processing circuit, a 3D scene based on an ultrasound signal, the 3D scene including a first anatomical feature;
positioning, by the processing circuit, a an abnormality of the first anatomical feature in the 3D scene based on the ultrasound signal;
generating and displaying, by the processing circuit, a resection volume on the first anatomical feature within the 3D scene based on the position of the abnormality;
determining, by the processing circuit, the resection volume interferes with a second anatomical feature;
adapting, by the processing circuit, the resection volume based on the determination that the planned resection volume interferes with the second anatomical feature; and
displaying an output image on a display screen of an ultrasound imaging system, the output image comprising the 3D scene including the adapted planned resection volume.

17. The method of claim 16, wherein the resection volume is automatically adapted based on a location of the first anatomical feature and the second anatomical feature.

18. The method of claim 16, wherein the method further comprises receiving, by the processing circuit, an abnormality margin, wherein the position and shape of the resection volume is based on a projection of the abnormality and the abnormality margin on a surface of the first anatomical feature.

19. The method of claim 16, wherein the resection volume is automatically adapted based on a location of the first anatomical feature and the second anatomical feature.

20. The method of claim 16, wherein determining that the planned resection volume interferes with the second anatomical feature is based on determining that the resection volume is within a margin of the second anatomical feature.
